(19) United States
(12) Patent Application Publication
(10) Pub. No.: US 2003/0068353 A1
(43) Pub. Date: Apr. 10, 2003

(54) Sustained release micro-porous hollow fiber and method of manufacturing the same

(75) Inventors: Frank L. Chen, Taoyuan (TW); Ya-Chin Wang, Hsinchu (TW)

Correspondence Address: SUGHRUE MION, PLLC, 2100 PENNSYLVANIA AVENUE, N.W., WASHINGTON, DC, 20037, US

(73) Assignee: INDUSTRIAL TECHNOLOGY RESEARCH INSTITUTE

(21) Appl. No.: 10/101,796

(22) Filed: Mar. 21, 2002

(30) Foreign Application Priority Data
Sep. 25, 2001 (TW) 90123549

(51) Int. Cl. A01N 25/34; A61K 7/42; A61K 9/70

(52) U.S. Cl. 424/411; 424/59; 424/443

(57) ABSTRACT

A sustained release micro-porous hollow fiber and method of manufacturing the same. The central duct and peripheral wall of the hollow fiber is filled with active agents and the open ends are enveloped with a thin layer of permeable resin or impermeable resin. The active agents fill in the central duct of the hollow fiber using a vacuum system to evacuate the air contained in the central duct. The open ends of the hollow fiber can be enveloped with a thin layer of impermeable or permeable resin. The open ends and the micro-pores of the wall of the hollow fiber can also be enveloped by a thin layer of permeable resin.

### BACKGROUND OF THE INVENTION

1. Field of the Invention

The present invention relates to a sustained release micro-porous hollow fiber and its derivatives. More particularly, the present invention relates to filling a central duct and peripheral wall of the hollow fiber with active agents and enveloping the open ends of the fiber with a thin layer of resin in order to sustain and/or control the release of the active agents.

2. Description of the Related Arts

Conventional sustained release systems are usually accomplished by preparing a microcapsule of a substrate, such as essence or medicine, and binding the microcapsule onto a material, such as fabric or leather, to sustain the release. The technology of preparing the conventional sustained release system is complicated.

The newly developed sustained release system of fiber contained medicine includes preparation using porous and non-porous fibers. An example of the former is in U.S. Pat. No. 4,801,458, which discloses a pharmaceutical preparation mainly composed of an adhesive compound layer having an arrangement of porous hollow fibers filled with medicines, and a support for supporting the adhesive compound layer. The hollow fibers have radially arranged open pores for releasing the medicines, however, the pores are produced unevenly, and this makes the medicines' release ineffective. Another example is in U.S. Pat. No. 5,538,735, which discloses a preparation of sustained release pharmaceuticals by non-porous hollow fibers. The pharmaceutical only releases from the the ends of the fibers, and are suitable for long fibers, theoretically.

Since the existing products have yet to achieve ideal release effect, there is still a need for a solution to produce more variable and release-controlled fibers, the primary object of the present invention.

### SUMMARY OF THE INVENTION

The present invention provides a better sustained release fiber and a method of manufacturing the same.

According to the present invention, a sustained release fiber is a micro-porous hollow fiber, wherein a central duct and peripheral wall of the hollow fiber are filled with a substrate with chemical or physical activity (hereafter as an active agent), and the open ends of the hollow fiber are enveloped with a layer of resin.

According to the present invention, the resin is a permeable or impermeable resin. When the resin is a permeable resin, the active agent is released through the resin-enveloped open ends and the micro-pores of the peripheral wall of the fiber. When the resin is an impermeable resin, the active agent is released only through the micro-pores of the peripheral wall of the fiber.

According to the present invention, the open ends and the micro-pores of peripheral wall of the fiber can be enveloped with a thin layer of permeable resin, and the sustained release can be achieved by releasing the active agent through the open ends and the micro-pores of the peripheral wall of the fiber.

According to the present invention, the method of manufacturing the sustained release fiber comprises: providing a hollow fiber with a central duct and micro-pores of the peripheral wall, immersing the hollow fiber in a solution of active agents, and drawing the air out from the central duct of the hollow fiber to conduct the active agents into the central duct of the hollow fiber. In the next step, the open ends and the micro-pores of the wall of the fiber can be enveloped by applying permeable or impermeable resin directly. Otherwise, the open ends and the micro-pores of the wall of the fiber can be immersed in a solution of resin to form a thin layer of resin, and then immersed in a heated solvent to make the resin permeable.

The present invention also provides applications of the sustained release fiber. In one preferred embodiment, the sustained release fiber in the present invention, including long fiber or short fiber, is mixed with other known fibers to produce a sustained release textile. In another preferred embodiment, the sustained release short fiber in the present invention is mixed with other known polymers to produce a sustained release plaster.

### BRIEF DESCRIPTION OF THE DRAWINGS

The present invention will be more fully understood and further advantages will become apparent when reference is made to the following description of the invention and the accompanying drawings in which:

FIG. 1 is a diagram showing the weight loss of porous hollow fibers with resins enveloped in example I. The total amount of the weight of the fiber is shown in mg at different days.

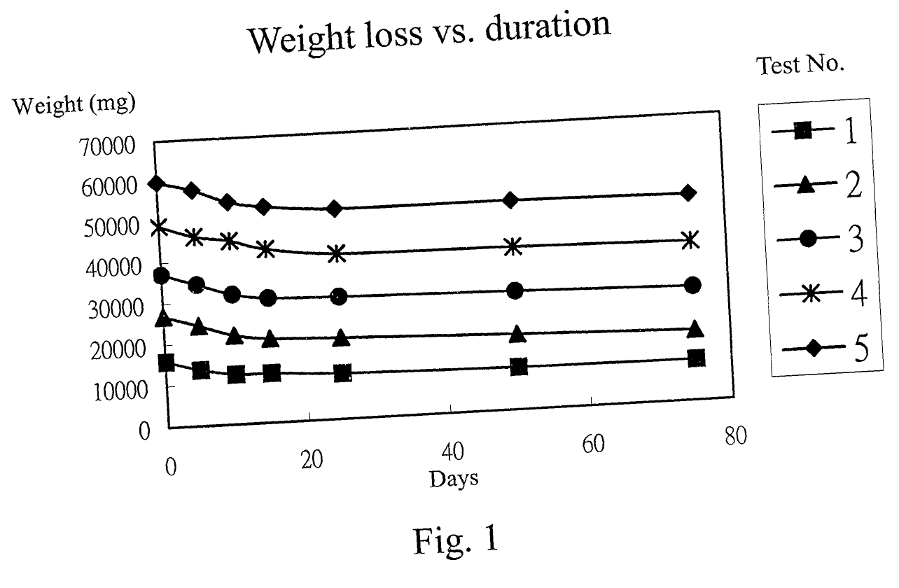

FIG. 2 is a diagram showing the average release rate of porous hollow fibers with resin enveloped in example I. The average release rate is shown in mg/day.

FIG. 3 is a diagram showing the weight loss of non-porous hollow fibers without resin enveloped in control I. The total amount of the weight of the fiber is shown in mg at different days.

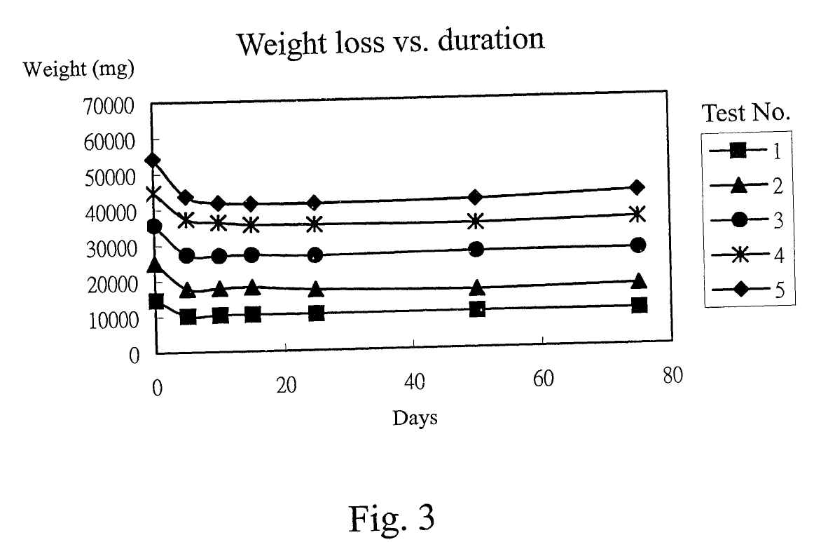

FIG. 4 is a diagram showing the average release rate of non-porous hollow fibers without resin enveloped in control I. The average release rate is shown in mg/day.

FIG. 5 is a diagram showing the weight loss of porous hollow fibers without enveloped ends in control II. The total amount of the weight of the fiber is shown in mg at different days.

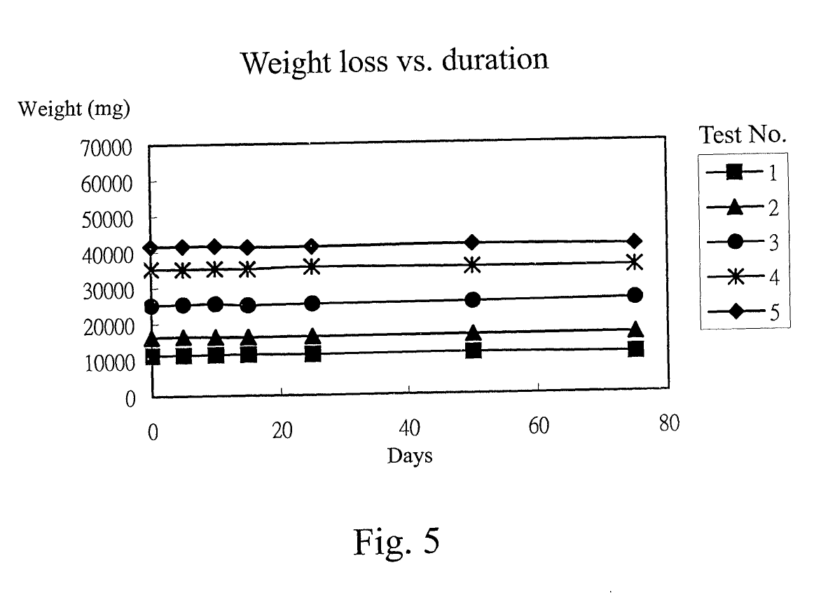

FIG. 6 is a diagram showing the average release rate of porous hollow fibers without enveloped ends in control II. The average release rate is shown in mg/day.

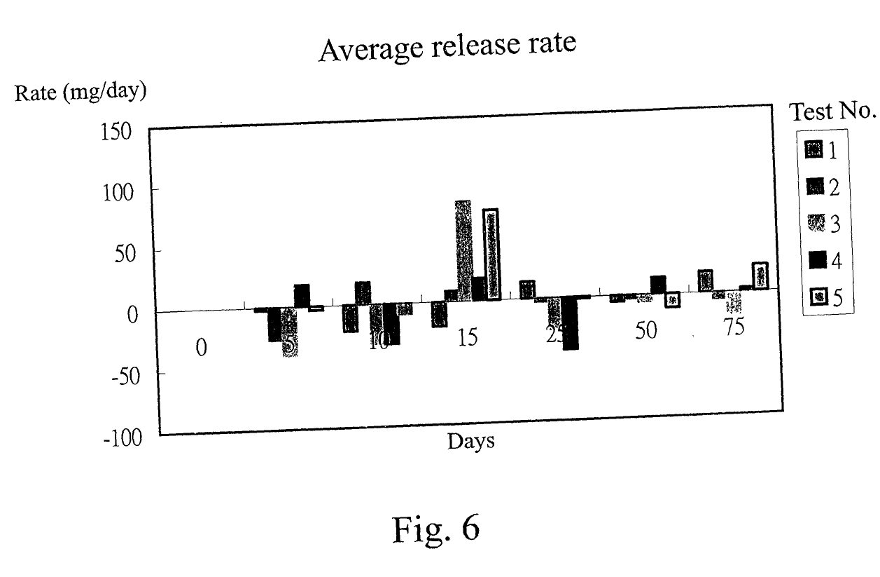

### DETAILED DESCRIPTION OF THE INVENTION

The release rate of active agents from fibers can be controlled by adjusting the environmental temperature and humidity; length, cross-section, number of pores, and diameter of pores of the hollow fiber; or the concentration of the active agents. In accordance with the present invention, the parameter of controlling the release rate is mainly determined by the number of micro-pores or the diameter of pores before or after enveloping. By controlling these parameters, the fiber and its derivatives have better efficiency of sustained release and better release control.

The micro-porous hollow fiber used herein includes, but is not limited to, polyester, rayon, cotton, Teflon, polyamide, cellulose, polyethylene, polypropylene, polystyrene, polyvinyl alcohol, polyvinyl chloride, polyacrylonitrile, polyurethanes, polyolefin, and the like, wherein the formation of micro-pores is known to be controlled during the method of manufacturing the fibers. The fiber is also available as Wellkey Filament®, a product of TEIJIN in Japan or HydroPore®, a product of the Chemical engineering division of the Industrial Technology Research Institute. The fiber can be a long or short fiber, for example, with a length of 5 to 150 mm. Single hollow fiber comprises at least one hole, or for example, 4 or 7 holes, and the hollow rate of the fiber is between 10 to 40%. The micro-pore of the fiber has a diameter of 0.01 to 5 μm.

The active agent used herein can be chosen according to the applied field, and is not limited. For example, the active agent can be an aromatic, a natural or synthetic essence, perfume, phytocide, or the like to keep the environment with a perfumed atmosphere. The active agent can be a curing agent, especially an agent administrated through skin such as antibiotics, antibody, steroid, vitamins, or an agent for curing epidermal, respiratory, and traumatic infection, or promoting blood clotting to prevent or treat skin diseases or conditions or diseases can be treated through skin. The active agent can also be a skin-whitening agent or cosmetic, for example, a UV-blocking agent, an infrared ray-blocking agent, a kind of cosmetic ingredients, a moisturizer, or the like. Another aspect of the active agent can be an insect repellent such as a mosquito repellent, a fly repellent, or insecticide for ant or other insect control. The active agent can be electrolytical, such as electrolytic agent for electric conduction or anti-static electricity. Other active agents include, but are not limited to, anti-bacterial agent, bacteriostat, anti-microbial agent, deodorizer, or water.

It is understood that the form of the active agent is not limited. Those with the ability to diffuse, evaporate, or sublimate the active molecule can be used. For the purpose of the present invention, the form of the active agent is preferably a liquid state which can be accomplished by dissolving the active agent into a hydrophilic or hydrophobic solvent to form a slurry, a suspension or an emulsion for filling the micro-porous hollow fiber.

In accordance with the present invention, a micro-porous hollow fiber is immersed in a solution of an active agent, and the fiber is then placed into a vacuum device to evacuate the air from the fiber by depressing into an atmosphere lower than the environmental atmosphere and to introduce the active agent into the fiber. The introduction of the active agent into the fiber can also be accomplished by pressurization. The vacuum device is known by those skilled in this art, and is also referred to U.S. Pat. No. 5,538,735.

After filling the active agent into the fiber, the micro-porous hollow fiber is enveloped with a layer of resin. Preferably, the resin used herein includes polyurethanes, acrylic resin, epoxy resin, or silicone resin which can be watery or oily.

The sustained release fiber, including long or short fibers, in the present invention can be mixed with any other known fibers to produce a sustained release textile. The form of the textile is woven, knit, or non-woven. In addition, the sustained release fiber, preferably short fiber, in the present invention can be mixed with any other known polymers to produce a sustained release plaster, for example, artificial leather, carpet, wallpaper, and the like. According to the present invention, the sustained release fiber is mixed with other fibers or polymers preferably by physical blending.

The sustained release fiber and its derivatives in the present invention can be applied in clothing such as materials for dress, shoes, or hat; home appliances; biomedical materials; automobile interior decoration; agricultural products; and the like. Their applications are widespread and valuable.

Without intending to limit it in any manner, the present invention will be further illustrated by the following examples.

### EXAMPLES

### Example I

Preparation of Sustained Release Fibers

A suitable amount of Hydropore® micro-porous hollow fibers were immersed in essence oil, and evacuated with 72 cm-Hg at 25° C. for 1 hour in a vacuum oven. After the evacuation, the fibers filled with essence oil were placed on a net support to drain redundant essence oil for 10 min. Two ends of the fibers were enveloped with optional polyurethanes and baked at 100° C. in an oven for 5 min. The stained release fibers in example I were obtained when the resin was heat-solidified.

Test of Release Rate

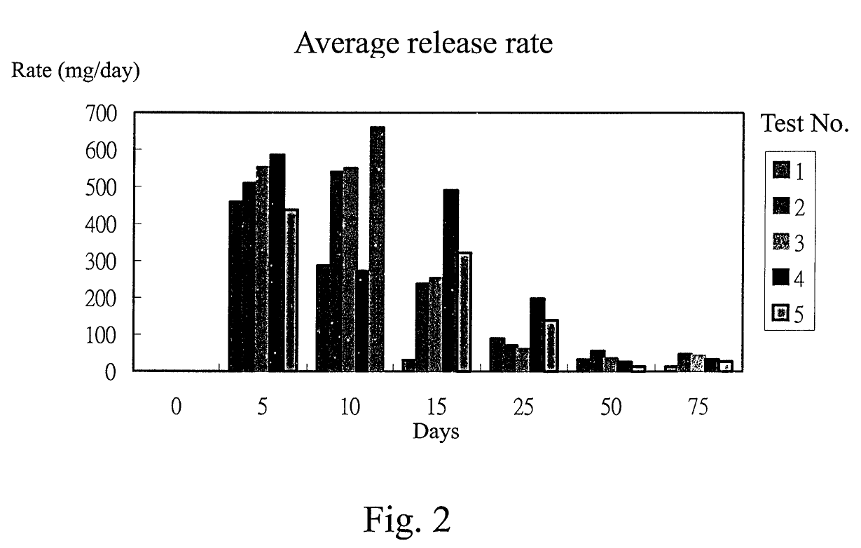

The sustained release fibers were placed at constant temperature and humidity, i.e. at 25° C., RH=60%. The duration and the weight change of the fibers were recorded. The process was repeated 5 times, and the results are shown in Table 1, Table 2 and FIG. 2.

TABLE 1
The total release test result of the sustained
release fiber in example I. (The total amount of the
sustained release fiber is shown in mg at different days.)

| Dura- | | | | | | | |
|---|---|---|---|---|---|---|---|
| tion | | | | | | | |
| Test | Duration (Days) | | | | | | |
| No. | 0 | 5 | 10 | 15 | 25 | 50 | 75 |
| 1 | 15842 | 13546 | 12108 | 11955 | 11056 | 10237 | 9901 |
| 2 | 26815 | 24261 | 21556 | 20365 | 19656 | 18254 | 17062 |
| 3 | 37054 | 34289 | 31535 | 30266 | 29654 | 28745 | 27659 |
| 4 | 48952 | 46021 | 44654 | 42196 | 40213 | 39546 | 38724 |
| 5 | 59762 | 57571 | 54266 | 52655 | 51265 | 50937 | 50265 |

TABLE 2
The average release test result of the sustained
release fiber in example I. (The average release rate was
shown by mg/day.)

| Duration | Duration (Days) | | | | | | |
|---|---|---|---|---|---|---|---|
| Test No. | 0 | 5 | 10 | 15 | 25 | 50 | 75 |
| 1 | 0 | 459 | 288 | 31 | 90 | 33 | 13 |
| 2 | 0 | 511 | 541 | 238 | 71 | 56 | 48 |
| 3 | 0 | 553 | 551 | 254 | 61 | 36 | 43 |
| 4 | 0 | 586 | 273 | 492 | 198 | 27 | 33 |
| 5 | 0 | 438 | 661 | 322 | 139 | 13 | 27 |

### Example II

Preparation of Sustained Release Fibers

A suitable amount of Hydropore micro-porous hollow fibers were immersed in essence oil, and evacuated with 72 cm-Hg at 25° C. for 1 hour in a vacuum oven. After the evacuation, the fibers filled with essence oil were placed on a net support to drain redundant essence oil for 10 min. The fibers were immersed in a DMF solution of polyurethane; therefore, the ends and the outer wall of the fibers were covered with polyurethane. The fibers covered with polyurethane were then bathed in water to exchange DMF solvent and water. After that, the fibers were baked at 100° C. in an oven for 5 min. The sustained release fibers with the ends and outer wall thereof enveloped by polyurethane in example II were obtained when the resin was heat-solidified.

Control I

Preparation of Non-Porous Fibers Without Enveloped Ends

A suitable amount of hollow fibers were prepared as Example I except the the ends of the fibers were not enveloped by resin. The hollow fibers without enveloped ends in Control I were thus obtained.

Test of Release Rate

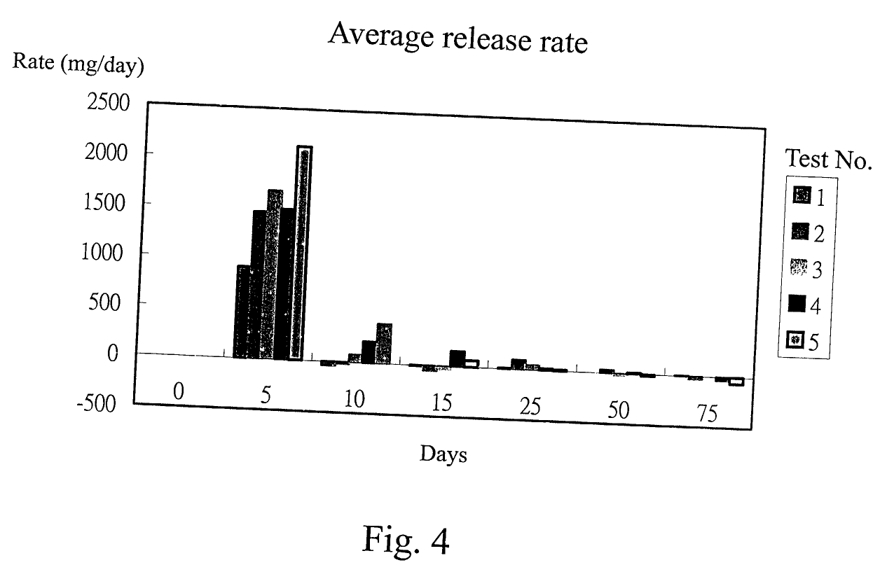

The fibers were placed at constant temperature and humidity, i.e. at 25° C., RH=60%. The duration and the weight change of the fibers were recorded. The process was repeated 5 times, and the results are shown in Table 3, Table 4 and FIG. 4.

TABLE 3
The total release test result of the fiber in Control I.
(The total amount of the fiber is shown in mg at different days.)

| Dura- | | | | | | | |
|---|---|---|---|---|---|---|---|
| tion | | | | | | | |
| Test | Duration (Days) | | | | | | |
| No. | 0 | 5 | 10 | 15 | 25 | 50 | 75 |
| 1 | 14655 | 10114 | 10298 | 10335 | 10255 | 10249 | 10165 |
| 2 | 24988 | 17656 | 17745 | 17988 | 17065 | 16325 | 17032 |
| 3 | 35675 | 27265 | 26891 | 27035 | 26564 | 27011 | 27036 |
| 4 | 44751 | 37254 | 36213 | 35464 | 35261 | 34925 | 35752 |
| 5 | 54262 | 43621 | 41659 | 41336 | 41234 | 41652 | 43250 |

TABLE 4
The average release test result of the fiber in Control I.
(The average release rate of the fiber is shown in mg/day.)

| Rate | Duration (Days) | | | | | | |
|---|---|---|---|---|---|---|---|
| Test No. | 0 | 5 | 10 | 15 | 25 | 50 | 75 |
| 1 | 0 | 908 | −37 | −7 | 8 | 0 | 3 |
| 2 | 0 | 1466 | −18 | −49 | 92 | 30 | −28 |
| 3 | 0 | 1682 | 75 | −29 | 47 | −18 | −1 |
| 4 | 0 | 1499 | 208 | 150 | 20 | 13 | −33 |
| 5 | 0 | 2128 | 392 | 65 | 10 | −17 | −64 |

Control II

Preparation of Porous Hollow Fibers Without Enveloped Ends

A suitable amount of porous hollow fibers were prepared as Example I except that the ends of the fibers were not enveloped by resin. The porous hollow fibers without enveloped ends in Control II were thus obtained.

Test of Release Rate

The fibers were placed at constant temperature and humidity, i.e. at 25° C., RH=60%. The duration and the weight change of the fibers were recorded. The process was repeated 5 times, and the results are shown in Table 5 and Table 6.

TABLE 5
The total release test result of the fiber in Control II.
(The total amount of the fiber is shown in mg at different days.)

| Dura- | | | | | | | |
|---|---|---|---|---|---|---|---|
| tion | | | | | | | |
| Test | Duration (Days) | | | | | | |
| No. | 0 | 5 | 10 | 15 | 25 | 50 | 75 |
| 1 | 11233 | 11245 | 11352 | 11452 | 11312 | 11446 | 11021 |
| 2 | 16212 | 16345 | 16254 | 16211 | 16241 | 16324 | 16471 |
| 3 | 25115 | 25312 | 25471 | 25065 | 25321 | 25474 | 25932 |
| 4 | 35214 | 35125 | 35289 | 35197 | 35624 | 35287 | 35219 |
| 5 | 41562 | 41578 | 41622 | 41255 | 41271 | 41562 | 41030 |

TABLE 6
The average release test result of the fiber in Control II.
(The average release rate is shown in mg/day.)

| Rate | Duration (Days) | | | | | | |
|---|---|---|---|---|---|---|---|
| Test No. | 0 | 5 | 10 | 15 | 25 | 50 | 75 |
| 1 | 0 | −2 | −21 | −20 | 14 | −5 | 17 |
| 2 | 0 | −27 | 18 | 9 | −3 | −3 | −6 |
| 3 | 0 | −39 | −32 | 81 | −26 | −6 | −18 |
| 4 | 0 | 18 | −33 | 18 | −43 | 13 | 3 |
| 5 | 0 | −3 | −9 | 73 | −2 | −12 | 21 |

Test Result:

Comparing examples and controls, examples maintained a release rate until day 15, however, the controls showed poor release ability. The fibers in Control I released most of the essence oil before day 5, and did not release any essence oil after day 10. The average release rate in Control 2 are mostly negative numbers. The fibers prepared in example I according to the present invention have significantly superior sustained release effect than those fibers prepared by other method.

While the invention has been particularly shown and described with the reference to the preferred embodiment thereof, it will be understood by those skilled in the art that various changes in form and details may be made without departing from the spirit and scope of the invention.

What is claimed is:

1. A sustained release fiber, comprising:
a hollow fiber comprising a central duct defined by an inner surface and the open ends, wherein the wall thereof comprises micro-pores,
an active agent filled in the central duct of the fiber, and
a thin layer of resin for enveloping the open ends of the fiber.

2. The sustained release fiber as claimed in claim 1, wherein the micro-porous hollow fiber is polyester, rayon, cotton, Teflon, polyamide, cellulose derivatives, polyethylene, polypropylene, polystyrene, polyvinyl alcohol, polyvinyl chloride, polyacrylonitrile, polyurethane, polyolefin or copolymers thereof.

3. The sustained release fiber as claimed in claim 2, wherein the micro-porous hollow fiber is long fiber.

4. The sustained release fiber as claimed in claim 2, wherein the micro-porous hollow fiber is short fiber.

5. The sustained release fiber as claimed in claim 1, wherein the active agent is aromatic, curing agent, insect repellent, moisturizer, anti-microbial agent, anti-viral agent, deodorizer, UV-blocking agent, or electrolyte.

6. The sustained release fiber as claimed in claim 5, wherein the active agent is in the form of liquid.

7. The sustained release fiber as claimed in claim 6, wherein the active agent is in the form of a slurry, a suspension, or an emulsion.

8. The sustained release fiber as claimed in claim 1, wherein the resin is heat solidified.

9. The sustained release fiber as claimed in claim 8, wherein the resin is watery or oily.

10. The sustained release fiber as claimed in claim 9, wherein the resin is polyurethane, acrylic resin, or silicone resin.

11. The sustained release fiber as claimed in claim 1, wherein the resin is permeable.

12. The sustained release fiber as claimed in claim 1, wherein the resin is non-permeable.

13. A sustained release fiber, comprising:
a hollow fiber comprising a central duct defined by an inner surface, and the open ends, wherein the wall thereof comprises micro-pores,
an active agent filling in the central duct of the fiber, and
a thin layer of permeable resin for enveloping the open ends and the micro-pores of the wall of the fiber.

14. The sustained release fiber as claimed in claim 13, wherein the micro-porous hollow fiber is polyester, rayon, cotton, Teflon, polyamide, cellulose derivatives, polyethylene, polypropylene, polystyrene, polyvinyl alcohol, polyvinyl chloride, polyacrylonitrile, polyurethane, polyolefin or copolymers thereof.

15. The sustained release fiber as claimed in claim 14, wherein the micro-porous hollow fiber is long fiber.

16. The sustained release fiber as claimed in claim 14, wherein the micro-porous hollow fiber is short fiber.

17. The sustained release fiber as claimed in claim 13, wherein the active agent is aromatic, curing agent, insect repellent, moisturizer, anti-microbial agent, anti-viral agent, deodorizer, UV-blocking agent, or electrolyte.

18. The sustained release fiber as claimed in claim 17, wherein the active agent is in the form of liquid.

19. The sustained release fiber as claimed in claim 18, wherein the active agent is in the form of a slurry, a suspension, or an emulsion.

20. The sustained release fiber as claimed in claim 13, wherein the resin is heat-solidified.

21. The sustained release fiber as claimed in claim 20, wherein the resin is watery or oily.

22. The sustained release fiber as claimed in claim 21, wherein the resin is polyurethane, acrylic resin, epoxy resin or silicone resin.

23. A method of manufacturing a sustained release fiber, comprising the steps of:
(a) providing a micro-porous hollow fiber composed of a central duct defined by an inner surface, and the open ends;
(b) immersing the fiber in a solution of an active agent;
(c) evacuating the air from the fiber to introduce the solution of the active agent into the central duct of the fiber; and
(d) enveloping the open ends of the hollow fiber with a permeable or non-permeable resin.

24. The method as claimed in claim 23, wherein the micro-porous hollow fiber is polyester, rayon, cotton, Teflon, polyamide, cellulose derivatives, polyethylene, polypropylene, polystyrene, polyvinyl alcohol, polyvinyl chloride, polyacrylonitrile, polyurethane, polyolefin or copolymers thereof.

25. The method as claimed in claim 24, wherein the micro-porous hollow fiber is long fiber.

26. The method as claimed in claim 24, wherein the micro-porous hollow fiber is short fiber.

27. The method as claimed in claim 23, wherein the step of evacuating the air of the fiber further comprises the step of placing the immersed fiber in a vacuum device, and decompressing to evacuate the air.

28. The method as claimed in claim 23, wherein the active agent is aromatic, curing agent, insect repellent, moisturizer, anti-microbial agent, anti-viral agent, deodorizer, UV-blocking agent, or electrolyte.

29. The method as claimed in claim 28, wherein the active agent is in the form of liquid.

30. The method as claimed in claim 29, wherein the active agent is in the form of a slurry, a suspension, or an emulsion.

31. The method as claimed in claim 23, wherein the resin is heat-solidified.

32. The method as claimed in claim 31, wherein the resin is watery or oily.

33. The method as claimed in claim 32, wherein the resin is polyurethane, acrylic resin, epoxy resin, or silicone resin.

34. A method of manufacturing a sustained release fiber, comprising the steps of:
(a) providing a micro-porous hollow fiber composed of a central duct defined by an inner surface, and the open ends;
(b) immersing the fiber in a solution of an active agent;
(c) evacuating the air of the fiber to introduce the solution of the active agent into the central duct of the fiber;
(d) bathing the fiber filled with the active agent into a solution of resin to envelope the open ends and the micro-pores of the wall of the fiber; and
(e) immersing the enveloped fiber in a solvent and heating to make the enveloping resin permeable.

35. The method as claimed in claim 34, wherein the micro-porous hollow fiber is polyester, rayon, cotton, Teflon, polyamide, cellulose derivatives, polyethylene, polypropylene, polystyrene, polyvinyl alcohol, polyvinyl chloride, polyacrylonitrile, polyurethane, polyolefin or copolymers thereof.

36. The method as claimed in claim 35, wherein the micro-porous hollow fiber is long fiber.

37. The method as claimed in claim 35, wherein the micro-porous hollow fiber is short fiber.

38. The method as claimed in claim 34, wherein the step of evacuating the air of the fiber further comprises the step of placing the immersed fiber in a vacuum device, and decompressing to evacuate the air.

39. The method as claimed in claim 34, wherein the active agent is aromatic, curing agent, insect repellent, moisturizer, anti-microbial agent, anti-viral agent, deodorizer, UV-blocking agent, or electrolyte.

40. The method as claimed in claim 39, wherein the active agent is in the form of liquid.

41. The method as claimed in claim 40, wherein the active agent is in the form of a slurry, a suspension, or an emulsion.

42. The method as claimed in claim 34, wherein the resin is heat-solidified.

43. The method as claimed in claim 42, wherein the resin is watery or oily.

44. The method as claimed in claim 43, wherein the resin is polyurethane, acrylic resin, epoxy resin, or silicone resin.

45. A sustained release textile, comprising:
a sustained release fiber as claimed in claim 1, and
one or a plurality of fibers mixed therewith.

46. The textile as claimed in claim 45, wherein the textile is in the structure of woven.

47. The textile as claimed in claim 45, wherein the textile is in the structure of non-woven.

48. The textile as claimed in claim 45, wherein the fiber is polyester, rayon, cotton, Teflon, polyamide, cellulose derivatives, polyethylene, polypropylene, polystyrene, polyvinyl alcohol, polyvinyl chloride, polyacrylonitrile, polyurethane, polyolefin or copolymers thereof.

49. A sustained release plaster, comprising:
a sustained release fiber as claimed in claim 1, and
one or a plurality of polymers mixed therewith.

50. The sustained release plaster as claimed in claim 49, wherein the fiber is short fiber.
